(19) United States
(12) Patent Application Publication
(10) Pub. No.: US 2007/0275481 A1
(43) Pub. Date: Nov. 29, 2007

(54) Methods For Detecting Half-Antibodies Using Chip Based Gel Electophoresis

(75) Inventors: Elena Vasilyeva, Swampscott, MA (US); Francine Brown, Amherst, NH (US); Matthias Kretschmer, Weston, MA (US); Peter Bove, Burlington, VT (US); Hans Fajardo, Wakefield, MA (US); Frederick R. Taylor, Milton, MA (US); Rohin Mhatre, Lexington, MA (US); Kazumi Kobayashi, Arlington, MA (US); Amy Dingley, Roslindale, MA (US)

Correspondence Address: WOLF GREENFIELD & SACKS, P.C., 600 ATLANTIC AVENUE, BOSTON, MA, 02210-2206, US

(73) Assignee: BIOGEN IDEC MA INC., CAMBRIDGE, MA (US)

(21) Appl. No.: 10/579,639

(22) PCT Filed: Nov. 23, 2004
(86) PCT No.: PCT/US04/41346

§ 371 Date: Jul. 2, 2007

Related U.S. Application Data
(60) Provisional application No. 60/525,108, filed on Nov. 24, 2003.

(51) Int. Cl. G01N 33/561; C07K 1/00; C07K 14/00; C07K 16/18; G01N 33/00

(52) U.S. Cl. 436/516; 436/86; 530/350; 530/387.1; 530/409

(57) ABSTRACT

The invention provides a method of using chip-based gel electrophoresis to determine the presence of polypeptides with selected disulfide linkage patterns, for example, completely formed tetrameric antibodies as compared to incompletely formed heterodimeric half-antibodies. The invention further features a kit, comprising a chip, and instructions for conducting the foregoing method. The methods and kits of the invention are amendable to high throughput applications for the monitoring of production and quality control of recombinant therapeutic antibodies.

### RELATED INFORMATION

The application claims priority to U.S. provisional patent application No. 60/525,108, filed on Nov. 24, 2003, the entire contents of which are hereby incorporated by reference.

The contents of any patents, patent applications, and references cited throughout this specification are hereby incorporated by reference in their entireties.

### BACKGROUND OF THE INVENTION

The immune response is a mechanism by which the body defends itself against foreign substances that invade it, causing infection or disease. This mechanism is based on the identification and binding of these foreign substances by antibodies. Once a substance is bound by an antibody, the substance is targeted for destruction.

Antibodies are composed of four polypeptides, two light chains and two heavy chains (L:H:H:L). Most antibodies contain disulfide bonds between the four polypeptide chains. Occasionally, so called half-antibodies occur, in which the disulfide bonds between the heavy chain polypeptides are not formed.

For some antibodies, such as the IgG4 class, 25-30% of IgG4 antibodies are produced as half-antibodies comprising a heavy and light chain, regardless of whether the molecules are produced recombinantly or naturally. For other antibody isotypes and sub-isotypes, half-antibody formation has been associated with aberrant protein forms. For example, half-antibody formation may be due to the structure of the hinge region, as in IgG4 antibodies, or with deletions in the heavy chain constant domains, as with antibodies produced by certain myelomas.

Half-antibodies are not associated with a distinct clinical syndrome, however, they have been identified in the serum and urine of patients with a variety of diseases such as multiple myeloma, plasma cell leukemia, and plasmacytoma. Half-antibodies, to some degree, are also produced by murine hybridomas and myelomas, and a byproduct of recombinant antibody production in both animal and bacterial cells. Many of these antibodies are potentially biologically less active when incomplete, and therefore have the potential to dilute the therapeutic effectiveness of a pharmaceutical preparation containing such half-antibodies. Accordingly, the ability to detect such molecules is desirable.

The typical method of determining the amount of half-antibodies in a sample, is by standard gel electrophoresis. This method is slow and error prone.

Accordingly, a need exists for an improved method for determining the presence of half-antibodies (as compared to completely formed antibodies), for monitoring and improving the quality control of antibody-based pharmaceuticals.

### SUMMARY OF THE INVENTION

The invention solves the foregoing problems of standard gel electrophoresis by providing improved methods for detecting the presence of a selected disulfide-linked polypeptide, for example, a half-antibody, as compared to a completely formed antibody. Typically, a completely formed antibody is a tetramer comprising two heavy chain polypeptides and two light chain polypeptides linked by disulfide bonds (L:H:H:L), whereas a half-antibody is a disulfide-linked light chain and heavy chain polypeptide (L:H) incompletely disulfide-linked or entirely uninked to a corresponding light chain and heavy chain polypeptide (H:L) (FIG. 11). Other exemplary molecules amenable to the methods described herein include any multimeric polypeptides having one or more disulfide bonds. The method of the invention is especially useful for determining the nature of disulfide linked multimeric polypeptides destined for use in human therapeutic applications, as well as, diagnostic and research applications.

The method employs chip-based gel electrophoresis which can readily, and rapidly, identify incompletely formed polypeptides, i.e., polypeptides having an incomplete number of disulfide linkage(s), for example, as compared to polypeptides having the desired number or placement of disulfide linkages. Two prominent examples include; antibodies, e.g., IgG4 antibodies which comprise four polypeptide chains held together by disulfide bonds, and multichain ligands, for example, growth factors.

Accordingly, the invention has several advantages which include, but are not limited to, the following:

a detection method of improved fidelity that can rapidly determine the amount of a selected disulfide-linked polypeptide, for example, a completely formed antibody as compared to an incompletely linked polypeptide, for example, a half-antibody, or undesired impurities;

a method for monitoring the quality control over the production, for example, recombinant production, of a polypeptide multimer (e.g., an antibody) for therapeutic use in, for example, a human subject; and

a kit which can be used with commercially available chips for conducting the methods of the invention.

Accordingly, in one aspect, the invention provides a method for detecting the presence of a IgG4 polypeptide having a selected disulfide linkage pattern in a sample, in which a sample containing a polypeptide having a selected disulfide linkage pattern is loaded onto a chip containing a channel having a separation medium effective to act as an obstacle to the migration of the polypeptide having a selected disulfide linkage pattern, and at least two electrodes disposed within the channel to induce an electric field. An electric field is applied across the separation medium of the chip, whereby a separation of the IgG4 polypeptide having a selected disulfide linkage pattern as compared to a IgG4 polypeptide not having the selected disulfide linkage pattern is achieved. The presence of the IgG4 polypeptide having a selected disulfide linkage pattern is determined, for example, the amount (e.g., percentage) of a completely formed (i.e., disulfide linked) antibody, half-antibody, or ratio thereof.

In a related aspect, the invention provides a method for detecting the presence of a polypeptide having a selected disulfide linkage pattern in a sample consisting of a mixture of polypeptide multimers having two or more polypeptide chains and containing, at least one disulfide linkage between the polypeptide chains. According to the method, a sample containing the mixture of polypeptide multimers is loaded onto a chip containing a channel having a separation medium effective to act as an obstacle to the migration of the polypeptide having a selected disulfide linkage pattern, and at least two electrodes disposed within the channel to induce an electric field. An electric field is applied across the separation medium of the chip whereby a separation of the polypeptide having a selected disulfide linkage pattern as compared to a polypeptide not having the selected disulfide linkage pattern is achieved. The presence of the polypeptide having a selected disulfide linkage pattern is detected. Such a peptide being, for example, a completely formed antibody or half-antibody.

In one embodiment, the sample of the above method contains an inhibitor of disulfide bond rearrangement that occurs as a result of polypeptide degradation, for example, a sulfhydryl alkylating reagent, such as iodoacetamide or N-ethylmaleimide (NEM), in an amount, for example, of between about 0.001, 0.003, 0.005, 0.007, 0.010, 0.030, 0.050, 0.070, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, and about 10.0 mM or more, and preferably, for example, in an amount of about 2 mM.

In a specific embodiment, the method is used to detect the presence of an IgG4 half-antibody, an IgG4 antibody, polypeptide impurities, or the ratio of any of the foregoing antibodies or polypeptides.

In a related embodiment, the antibody is a monoclonal antibody, for example, a humanized antibody, in particular, an anti-integrin antibody (e.g., an anti-alpha-4-beta-1 (VLA4) antibody, anti-alpha-4-beta 7 (VLA-7) antibody, or an antibody that binds both VLA-4 and VLA-7, e.g., natalizumab).

In another embodiment, the sample comprises a polypeptide (e.g., a polypeptide with a disulfide linkage, e.g., an antibody of ligand) at a concentration of about 1 ug/ml to about 500 ug/ml (including any range of polypeptide concentration therein, e.g., 1-10 ug/ml; 10-100 ug/ml; 100-500 ug/ml; 500-1000 ug/ml; and 1000-5000 ug/ml, as well as any overlapping range or narrower range of the foregoing polypeptide concentration ranges).

In another embodiment, the method detects a polypeptide that is a ligand having a selected disulfide linkage pattern, for example, completely formed as compared to incompletely formed.

In another embodiment, the above methods are suitable for detecting a selected disulfide linkage in a polypeptide that is produced recombinantly.

In another embodiment, the above methods are suitable for detecting a selected disulfide linkage in a polypeptide that is produced recombinantly.

In a related embodiment, the method is suitable for detecting polypeptide impurities in the presence of the foregoing ligand, antibody, half-antibody, or disulfide linked polypeptide.

In a related embodiment, the method detects a selected disulfide linkage in polypeptide that is isolated from the growth medium of a cell culture.

In a related embodiment, the separation medium used in the above method is a gel polymer, for example, a non-reducing gel polymer.

In a related embodiment, the migration of the polypeptide in the separation medium is detected using a fluorescence detector.

In a related embodiment, the polypeptides are separated according to their molecular weights.

In a related embodiment, the separation mechanism comprises isoelectric focusing, in which the molecules are separated based on their isoelectric point.

In another embodiment, the chip used in the separation method comprises a precast gel polymer.

In yet another embodiment, the invention provides a kit for detecting the presence of a polypeptide having a selected disulfide linkage pattern. The kit contains a chip, and instructions for carrying out the method described herein for determining the presence of a polypeptide with a selected disulfide pattern, for example, an antibody, half-antibody, or polypeptide impurity, or ratio of any of the foregoing antibodies or polypeptides.

In a still another embodiment, the invention provides a kit for determining the purity of a therapeutic polypeptide having a selected disulfide-linkage pattern. The kit contains a chip and instructions for carrying out the method for determining the amount of polypeptide with a selected disulfide linkage pattern, for example, an antibody, half-antibody, or polypeptide impurity, or ratio of any of the foregoing antibodies or polypeptides.

In a related embodiment, the invention features a kit for determining the presence or purity of a polypeptide with a selected disulfide-linkage pattern consisting of a chip, instructions for carrying out the assay for determining the amount of polypeptide with a selected disulfide-linkage pattern, and one or more of the following components such as, separation medium, non-reducing buffer, protein dye, formulation buffer, or a means for inducing an electric field through a separation medium.

In another aspect, the invention provides a method of inhibiting disulfide bond rearrangement that occurs as a result of polypeptide degradation, for example, degradation that occurs when the polypeptide is exposed to denaturing conditions and/or heat, the method comprising incubating the polypeptide with a sulfhydryl alkylating agent, such as iodoacetamide or N-ethylmaleimide (NEM).

In one embodiment, the sulfhydryl alkylating reagent is N-ethylmaleimide (NEM) and present, for example, in an amount between about 1 mM to about 10 mM.

In another aspect, the invention provides a composition containing a polypeptide, for example, a polypeptide multimer, and an inhibitor of polypeptide degradation, wherein the inhibitor is a sulfhydryl alkylating agent, such as iodoacetamide or N-ethylmaleimide (NEM).

In one embodiment, the polypeptide multimer is an antibody or half-antibody, for example, an IgG4 antibody, and, in a specific embodiment, an anti-integrin antibody.

Other features and advantages of the invention will be apparent from the following detailed description and claims.

### BRIEF DESCRIPTION OF THE DRAWINGS

FIG. 1 depicts a chromatograph showing the sizing accuracy (by migration time) of the method on an intact antibody (anti-integrin antibody) and corresponding half-antibody, as a function of sample concentration.

FIG. 2 depicts chromatographs showing increasing amounts (panels A-D) of an antibody (anti-integrin antibody), as compared to a control, to determine the linear dynamic range of the assay.

FIG. 3 depicts the linearity of the method by plotting the relative concentration (calculated by determination of fluorescence by comparison to a marker of known concentration running with the sample) versus the actual sample concentration (determined by A₂₈₀ readings) of an antibody (anti-integrin antibody), using the concentration range of 100 μg/ml to 5000 μg/ml.

FIG. 4 depicts the linearity of the method by plotting the relative concentration versus the sample concentration of an antibody (anti-integrin antibody), using the concentration range of 100 μg/ml to 2000 μg/ml.

FIG. 5 depicts the linearity of the method by plotting the relative concentration versus the sample concentration of half-antibodies using the concentration range of 100 μg/ml to 5000 μg/ml.

FIG. 6 depicts the linearity of the method by plotting the relative concentration versus the sample concentration of half-antibodies using the concentration range of 100 μg/ml to 2000 μg/ml.

FIG. 7 depicts the correlation between the relative and absolute concentration of sample determined by software analysis and the real sample concentration based on absorbance readings.

FIG. 8 depicts a graph showing the % of half-antibody at sample protein amounts ranging from 0.1 μg to 0.5 μg.

FIG. 9 depicts a graph showing the % of half-antibody at sample protein concentrations ranging from 10 μg/ml to 4900 μg/ml used to determine optimum sample concentration.

FIG. 10 depicts a electrothermogram used to detect the lowest detectable concentration of half-antibody using the assay.

FIG. 11 depicts the structure of an antibody with intrachain and interchain disulfide linkages indicated. Typically, IgG4 intrachain disulfide linkages occur between light chain residues 23 and 92; 138 and 198; and between heavy chain residues 22 and 96; 145 and 201; 262 and 322; and 368 and 426. IgG4 inter-heavy chain disulfide linkages typically occur at residues 227 and 230.

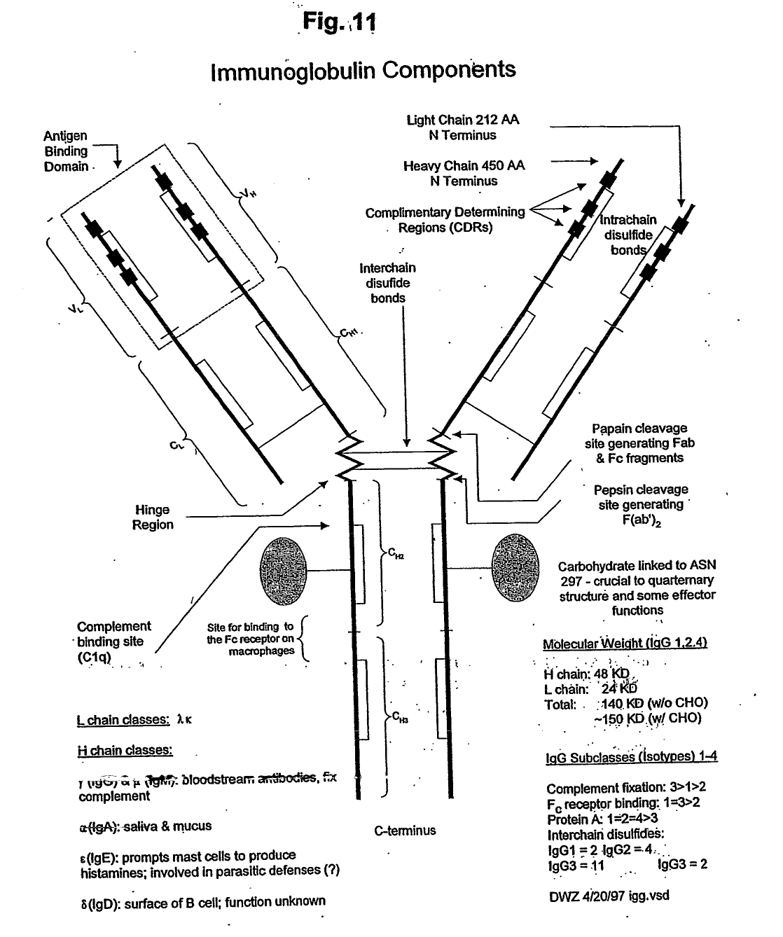

FIG. 12 depicts a electrothermogram of an exemplary sample having known concentrations of “impurities” as represented by marker proteins for determining the level of detection of impurities using the chip-based gel electrophoresis method (see also Table 7 and text).

FIG. 13 depicts a digital image of SDS-PAGE analysis of exemplary test samples and the contaminants that can be detected using the chip-based gel electrophoresis method in a recombinant antibody preparation. The arrows pointing to the right indicate known contaminant protein bands (sample/lane 1 contains trypsin inhibitor, sample/lane 2 contains ovalbumin, sample/lane 3 contains β-galactosidase, and sample/lane 4 contains a control standard). The arrows pointing to the left indicate contaminate proteins from the recombinant sample and “HC” and “LC” indicate, respectively, the heavy and light chains of the recombinant antibody (i.e., natalizumab).

FIG. 14 depicts electrothermograms using the chip-based method of representative samples containing contaminant proteins previously examined by SDS-PAGE (FIG. 13). Panels appearing from top to bottom correspond to lanes 1-4 of the gel image shown in FIG. 13. In each case, the marker protein (i.e., trypsin inhibitor, top panel; ovalbumin, second panel down; β-galactosidase, third panel down) is easily detected as a peak (see arrows) which does not occur in the control standard (bottom panel).

FIG. 15 shows overlapping electrothermograms of an antibody sample prepared in the presence and absence of N-ethylmaleimide (NEM) as analyzed by reverse-phase HPLC. Results demonstrate a lower percentage of half-antibody products in the sample treated with NEM then the sample without NEM (4.5% versus 9.2%; see arrow labeled “Half-molecule”) as well as significantly fewer by-products (indicated by arrows). Samples were denatured and heated at 100° C. for 3 min in the presence or absence of 2 mM NEM.

FIG. 16 shows a digital image of a an antibody sample prepared in the presence and absence of N-ethylmaleimide (NEM) when analyzed by non-reducing SDS-PAGE. Results demonstrate a lower percentage of half-antibody products in the sample treated with NEM then the sample without NEM (7.5% versus 13% when quantitated using ImageQuant software; see also arrow labeled “Half-Ab”) as well as significantly fewer by-products indicated by additional arrows). Samples were denatured and heated at 100° C. for 3 min in the presence or absence of 2 mM NEM.

FIG. 17 shows a graph indicating that the effect of heating time on the percentage of half-antibody as determined by the chip-based method described herein. Results demonstrate that samples in the presence of NEM (2 mM) (open symbols) are resistant to heat-mediated degradation over time (i.e., 1, 2, 3, 4, and 5 minutes) as compared to samples without NEM (closed symbols).

FIG. 18 shows a graph indicating the ability of NEM to inhibit protein degradation (as a function of percent half-antibody observed) over a range of NEM concentrations (i.e., protein samples had from 0 to 10 mM NEM).

FIG. 19 shows the chemical structure of two exemplary sulfhydryl alkylating agents suitable for inhibiting protein degradation. The structure of iodoacetamide is shown in the upper panel, the structure of N-ethylmaleimide (NEM) is shown in the lower panel.

### DETAILED DESCRIPTION OF THE INVENTION

In order to provide a clear understanding of the specification and claims, the following definitions are conveniently provided below.

Definitions

As used herein the term a “polypeptide having a selected disulfide linkage” includes a half-antibody where the selected disulfide linkage occurs between the light chain polypeptide and heavy chain polypeptide and not between the heavy chain polypeptides.

The term “chip” includes any solid substrate having a means for containing a separation medium that can have an electric field applied across the medium such that it can be used to separate macromolecules, e.g., multimeric polypeptides, such as antibodies and half-antibodies.

The term “separation medium” includes any compound, for example, polymer gel, that can be used to differentially separate macromolecules, e.g., multimeric polypeptides, such as antibodies and half-antibodies and is generally understood to comprise an appropriate buffer solution.

The term “half-antibody” includes antibodies in which, the inter-heavy chain disulfide bond(s) are absent, such that a single light chain polypeptide and a single heavy chain polypeptide form unconnected to a corresponding single light chain polypeptide and a single heavy chain polypeptide.

The term “IgG4 class” includes a subclass of IgG immunoglobulins that are produced during a secondary immune response and are most commonly found in the blood. These IgG antibodies typically contain the γ4 heavy chain.

The term “non-reducing” refers to conditions under which disulfide-bonds (e.g., disulfide linkage(s) are preserved. Specifically, conditions under which disulfide bonds remain intact and are not converted to free sulfhydryls

The term “isoelectric focusing” includes methods in which macromolecules will migrate and focus in a pH gradient, established by applying an electric charge to a solution of carrier ampholytes, according to their isoelectric point (pI).

The term “integrin” includes any polypeptide representative of the large family of transmembrane proteins, so named, which are involved in the adhesion of cells to the extracellular matrix. Accordingly, the term an “anti-integrin antibody” is an antibody that binds to such a molecule. Examples of anti-integrin antibodies include such antibodies as anti-alpha-4-beta-1 (VLA-4) antibodies, anti-alpha-4-beta 7 (VLA-7) antibodies, and antibodies that bind both VLA-4 and VLA-7, e.g., natalizumab (see, for example, U.S. Pat. No. 6,033,665, and U.S. Pat. No. 5,840,299.

The term “inhibitor”, for example, “inhibitor of disulfide bond rearrangement” includes any agent suitable for inhibiting disulfide bond rearrangement as a result of polypeptide degradation, in particular, the formation of polypeptide fragments or other undesired by-products in a polypeptide sample, for example, a polypeptide multimer sample, more particularly, an antibody sample where the by-products can be, for example, half-antibodies, or antibody or half-antibody polypeptide fragments. Preferred inhibitors of disulfide bond rearrangement that occurs as a result of polypeptide degradation include sulfhydryl alkylating agents, for example, iodoacetamide and N-ethylmaleimide.

The term “disulfide bond rearrangement” includes any undesired intra- or inter-chain disulfide bond that can form in a polypeptide, e.g. a polypeptide multimer such as an antibody. Typically, such disulfide bond rearrangements occur as a result of protein degradation which allows, for example, the exposure of reactive groups, e.g., thiols, which can interact with other regions of the polypeptide(s).

### DETAILED DESCRIPTION

A method has been developed to detect the presence of a polypeptide having a selected disulfide-linkage, for example an antibody, using chip-based gel electrophoresis. The method is especially well suited for detecting the presence, absence, or relative amounts (ratios) of completely formed antibody (which is a disulfide-linked tetramer), for example an IgG4 antibody, as compared to an incompletely formed antibody, for example, a half-antibody, lacking inter-heavy chain disulfide linkage(s) (which is a disulfide-linked heterodimer). In addition, the method can be used to detect the presence of impurities in a sample, e.g., polypeptide impurities. The method typically uses an Agilent 2100 Bioanalyzer in combination with the Protein 200 (or 200 Plus) LabChip kit and Protein 200 assay software (available from Agilent Technologies), although similarly conformed hardware, reagents, and software may be used.

As proof of principle, the method of the invention described herein uses test samples obtained during the production of a recombinant antibody for therapeutic use, specifically, an anti-integrin antibody, although it is understood that the method can be equally applied to any antibody sample whether naturally-derived (e.g., from serum), produced by a cell line (e.g., a hybridoma), or produced in a transgenic organism.

The method offers several advantages over current SDS-PAGE method, for example, chip-based gel electrophoresis is highly automated, and is easy to use. In contrast to the conventional SDS-PAGE, no additional manual staining or destaining steps are required. In addition, automation allows for near real time analysis of a polypeptide product at different stages during the production process, e.g., from clonal selection of a cell line (e.g., from a cell bank), to cell culture expansion, and final production phase.

The method is performed by obtaining a sample comprising a polypeptide having a selected disulfide linkage pattern, for example, a multimeric polypeptide held together by one or more disulfide bonds or linkages, for example an antibody or half-antibody, and loading or contacting the sample with a separation medium or gel, for example a organic polymer, which is confined to a opening or channel on a solid substrate, such as a chip. The separation medium or gel is then subjected to an electric field (i.e., a current is applied to the gel) such that the polypeptide molecules migrate through the separation medium based on molecular weight. The migration of the polypeptide through the separation medium or gel being performed under conditions which preserve the disulfide linkage pattern (or disulfide bonds) of the polypeptide (e.g., non-reducing conditions). Accordingly, polypeptides having different disulfide linkage patterns can be distinguished, and in particular, multimeric polypeptides, such that tetrameric antibodies can be distinguished from heterodimeric half-antibodies.

In one embodiment, the actual detection of the migration pattern of the polypeptides having selected disulfide linkages is carried out using a dye (e.g., a fluorescent dye) which can interact with the polypeptide and be monitored visually, e.g., using a optical detection device (e.g., a fluorescence reader).

In another embodiment, the polypeptide sample, for example, polypeptide multimer, such as an antibody, is prepared using an agent that inhibits disulfide bond rearrangement that occurs as a result of polypeptide degradation, e.g., polypeptide degradation caused or accelerated by heat and/or denaturation conditions. The inhibitor of polypeptide degradation can be a sulfhydryl alkylating agent, for example, iodoacetamide or N-ethylmaleimide (NEM).

In another embodiment, a composition comprising a polypeptide further comprises or is exposed to an inhibitor of polypeptide degradation, e.g., a sulfhydryl alkylating agent, for example, iodoacetamide or N-ethylmaleimide (NEM). Conditions, reagent concentrations, detection dyes, and appropriate apparatus for conduction gel electrophoresis are well known in the art. Moreover, there exist numerous variations on the buffer conditions and detection reagents which may be adapted to the methods of the invention. Accordingly, the invention has the advantage of being conveniently incorporated into established protocols without the need for extensive re-optimization. In addition, the methods of the invention may be used for monitoring the nature of any polypeptide having a disulfide linkage.

In one particular embodiment, the methods of the invention are conducted, as noted above, using an Agilent 2100 Bioanalyzer together with the Protein 200 (or 200 Plus) LabChip Kit and the dedicated Protein 200 assay software which provides detailed data of the polypeptide size and concentration for up to 10 samples within 45 minutes. Software calculations are performed which provide the molecular weight (based on amount of fluorescence detected over time) for each polypeptide detected in the sample, relative polypeptide concentrations corresponding to each other polypeptide detected, and amount (percentage) of each polypeptide compared with the total relative polypeptide concentration in the sample. The method allows for an immediate determination of the amount (percentage) of half-antibody molecules in a given sample without the additional procedures necessary for conducting SDS-PAGE. The method also allows for the detection of impurities, e.g., polypeptide impurities.

The data presented in the examples below demonstrate the high precision of the method, particularly for determining the amount of completely formed antibodies relative to incompletely formed antibodies, i.e., half-antibodies, as well as impurities. Moreover, the method of the invention using a chip-based gel electrophoresis approach is an illustration of the power of integrating multiple operations, on a chip, e.g., staining, separating, and diluting protein samples, thereby providing a superior approach over conventional gel electrophoresis methods.

The invention also provides kits for the convenient practice of the methods of the invention. In one embodiment, the invention provides a kit for detecting the presence of a polypeptide having a selected disulfide linkage pattern comprising, a chip and instructions for carrying out the methods as described herein. The kit may also contain at least one other component such as separation medium, non-reducing buffer, protein dye, formulation buffer, and means for inducing an electric field through a separation medium.

In a preferred embodiment of the invention, any of the foregoing kits may be further designed, packaged, or provided with instructions such that the kit may be conveniently used with a separation medium, non-reducing buffer, protein dye, chip and/or means for inducing an electric field through a separation medium, such as are available from, e.g., Agilent Technologies (see, e.g., U.S. Pat. No. 6,254,754).

It is understood that other commercially available separation media (gels), detection agents (dyes), detectors, chips, and apparatuses for inducing an electric field, for carrying out the methods disclosed herein, are also encompassed by the invention (see, e.g., U.S. Pat. Nos. 6,176,990; 6,261,430; 5,750,015; 5,449,446; and 5,427,663).

The methods of the invention are applicable to a variety of uses including, bioproduction, research, diagnostic applications, and forensic science.

Bioproduction

The methods and kits of the invention have a variety of applications in the bioproduction of a polypeptide having a disulfide linkage. Indeed, antibodies (but also multimeric ligands) are a favored class of therapeutic polypeptides being commercially produced which are multimeric polypeptides linked by disulfide bonds.

Accordingly, the methods of the invention, as exemplified herein, are readily applied to the monitoring or quality control (QC) of any relevant stage of the bioproduction of a disulfide linked polypeptide, e.g., clonal selection from a clone bank, cell culture expansion, and small and large scale production, e.g., using biofermentors. Because the methods of the invention are rapid and can be automated, near real time analysis can be performed during production, and if desired, used to alter production parameters to influence the quality of production output.

Research Applications

The methods and kits of the invention have a variety of research applications. For example, they are useful for any research application in which an analysis must be performed rapidly or on limited amounts of a sample containing a polypeptide with a disulfide linkage, e.g., a multimeric polypeptide, such as an antibody or ligand.

Other applications of the methods of the invention for research uses will be readily apparent to those skilled in the art.

Diagnostic Applications

The methods and kits of the invention are useful in a variety of diagnostic applications, such as the detection of inappropriate unlinked polypeptides (e.g., half-antibodies) in a patient.

The methods and kits of the invention described herein may also be used to detect or characterize antibodies and/or half-antibodies associated with diseases, e.g., genetic disorders or cellular disorders, such as cancer.

Forensic Applications

Forensic science is concerned with the scientific analysis of evidence from a crime. Forensic biology applies the experimental techniques of molecular biology, biochemistry, and genetics to the examination of biological evidence for the purpose, for example, of positively identifying the perpetrator of a crime. Typically, the sample size of such biological evidence (e.g. blood) is small yet contains a sufficient amount of a polypeptide, for example, an antibody, capable of being detected according to the method of the invention.

Accordingly, the improved chip-based gel electrophoresis techniques of the invention may be used to detect polypeptides, e.g., identifying antibodies, from even small biological samples.

The following examples are included for purposes of illustration and should not be construed as limiting the invention.

### Exemplification

Throughout the examples, the following materials and methods were used unless otherwise stated.

### Materials and Methods

In general, the practice of the present invention employs, unless otherwise indicated, conventional techniques of chemistry, molecular biology, recombinant DNA technology, immunology (especially, e.g., immunoglobulin technology), and standard techniques in electrophoresis. See, e.g., Sambrook, Fritsch and Maniatis, Molecular Cloning: Cold Spring Harbor Laboratory Press (1989); Antibody Engineering Protocols (Methods in Molecular Biology), 510, Paul, S., Humana Pr (1996); Antibody Engineering: A Practical Approach (Practical Approach Series, 169), McCafferty, Ed., Irl Pr (1996); Antibodies: A Laboratory Manual, Harlow et al., C.S.H.L. Press, Pub. (1999); Current Protocols in Molecular Biology, eds. Ausubel et al., John Wiley & Sons (1992). Bousse et al., Protein Sizing on a Microchip, Anal. Chem. 73, 1207-1212 (2001); Knapp et al., Commercialized and Emerging Lab-on-a-Chip Applications; In: Proceedings of the μTAS 2001 Symposium, Ramsey, J. M. & van den Berg, A., 7-10 (2001); and Mhatre et al., Strategies for locating disulfide bonds in a monoclonal antibody via mass spectrometry, Rapid Commun. Mass Spectrom, 13 (24) 2503-2510 (1999); Sey et al., Limited Proteolysis of the Third Component of Human Complement C3 by Heat Treatment, J. Biochein. 89:659-664 (1981); and Seya et al., Heat-Induced Thio-disulfide Interchange Reaction on the Third Component of Human Complement C3, J. Biochem. 103: 792-796 (1988).

Equipment and Samples

Separation and detection of antibody samples was done using the Agilent 2100 Bioanalyzer instrument in combination with the Protein 200 (or 200 Plus) LabChip kit and the dedicated Protein 200 assay software. The Bioanalyzer uses epifluorescent detection with a 10-mW semiconductor laser that emits at 630 nm. The instrument also contains 16 individually programmable high-voltage supplies.

TABLE 1
Materials, Equipment, and Computer Systems:

| Item description | Manufacturer and Model # |
|---|---|
| Agilent 2100 Bioanalyzer | Agilent Technologies G2943AA |
| Agilent 2100 Bioanalyzer Software (revisions | Agilent Technologies |
| A.02.01 and A.02.11) | |
| Protein 200 (or 200 Plus) LabChip ®Kit | Agilent Technologies (cat.# 5065-4430) |
| (reagents, 25 LabChips, 1 cleaning chip, 1 | |
| syringe and reagent kit guide) | |
| Chip Priming Station used to load gel matrix | Agilent Technologies (cat# 5065-4401) |
| into chip. | |
| Protein 200 Ladder and Upper Marker to use | Agilent Technologies (cat# 5065-4434) |
| with the Protein 200 and 200+ LabChip Kit | |
| Materials and Equipment to perform non- | Standard Techniques |
| reducing SDS-PAGE | |
| Densitometer and ImageQuant Software | Molecular Dynamics |
| NEM (N-Ethylmaleimide) | Fluka BioChemika (cat# 04260) |
| NEM (N-Ethylmaleimide) | Pierce (cat# 23030) |
| NEM (N-Ethylmaleimide) | Calbiochem (cat# 34115) |
| Iodoacetamide | Fluka BioChemika (cat# 57670) |

Sample Preparation

Protein samples were diluted as needed using formulation buffer (e.g., PBS or 10 mM sodium phosphate, pH 6.0, 140 mM NaCl, 0.02% Tween-80) and denatured by mixing in a 2:1 ratio of denaturing buffer, and heated to 100° C. for 5 min. Denaturing buffer is provided with Agilent Protein 200 Assay kit and contains 4% SDS and 290 μg/ml myosin internal marker. Following denaturing, the samples for the experiments were diluted 1:15 in a 10% solution of lower marker dye from Agilent Technologies (excitation/emission wavelength 650/680 nm) in deionized water. Optimum protein concentration was determined to be 3000 μg/ml. Over ten different, representative samples of an anti-integrin antibody produced recombinantly in NSO cells, using standard techniques, were used in the assays that follow. Alternatively, to limit polypeptide degradation upon, e.g., exposure to heat and/or denaturation conditions, polypeptide samples where prepared by mixing in a 2:1 ratio with denaturing buffer that contained NEM (or iodoacetamide), and were heated to 100° C. (in boiling water) for 3 min. Denaturing buffer as provided in the Agilent Protein 200 Plus Assay kit containing 4% SDS and protein upper and lower internal markers, was used. Inhibitors such as NEM were added to a final concentration of 15 mM (unless otherwise noted). Following denaturation, the samples were diluted 1:15 in deionized water.

Chip Preparation (Chip Priming)

For all separation experiments every channel in the glass chip was filled by loading the sieving matrix into the right upper well labeled “G” and applying pressure for 1 min by following the standard procedure described in the Agilent Reagent kit guide (2000) (incorporated by reference). The sieving matrix used is a polymer based on polydimethylacrylamide at 3.25% in a Tris-Tricine buffer at pH 7.6 (120 mM Tricine, 42 mM Tris), containing 0.25% SDS (8.7 mM final concentration) and 4 μM of the same dye used as a lower marker (“Agilent” dye). The sieving matrix is prepared according to the standard procedure using the reagent provided in the kit. All four wells labeled “G” are filled with this solution. The SDS dilution well (labeled “DS”) contains only the sieving matrix and the Tris-Tricine buffer. Protein samples (total volume 6 μl) are applied to all remaining wells on the chip except the well next to G on the bottom. This well (with the ladder mark) is filled with the protein ladder which includes 7 proteins with molecular weights from 14 to 210 kD.

Data Capture and Analysis

In the chip, electrophoresis moves each sample sequentially from its well to the central channel. As the samples move down the central channel they separate by size, finally passing the laser that excites the fluorescent dye bound to the sample. Data capture and analysis are performed with Agilent Technologies 2100 Bioanalyzer Software (revision A.02.01). The software analyzes data based on fluorescence intensity versus time. Quantitating the concentration and protein sizing are achieved by comparing against a sizing ladder and internal standards (“markers”) which are run with each sample. The data are presented as a gel-like image, an electropherogram, and in a tabular format (combined result table) which includes data on migration time, fluorescence intensity (as “Corr.area”), size, relative concentration, absolute concentration (option), and “% total”.

The electropherogram from the Agilent 2100 Bioanalyzer visualizes the separation of the proteins according to their molecular weight (kD). The Protein 200 ladder is run on each chip from a designated ladder well. Following the analysis of the Protein 200 ladder, the software generates a calibration curve of the migration time versus the molecular weight of each protein in the ladder. This calibration curve is then used to determine the size of each of the detected proteins in the 10 samples. The lower and upper markers, which are run with each of the 10 samples, correct for small drifts in migration time and ensure accurate sizing. The software automatically performs a sizing based on alignment with internal markers and an external protein standard. The determination of the molecular weight is based on the measured electrophoretic migration time given in seconds.

The relative protein concentrations (μg/ml) were determined based on measuring peak areas and comparing them to the internal standard (the upper marker) of known protein concentration. The % Total is calculated based on the relative concentrations.

The results for each sample are viewed in real-time when detection is completed. The first result is available in seven minutes with each subsequent analysis following in 2-minute intervals. The results are displayed in a tabular format, a gel-like image, and an electropherogram for each sample.

Each electropherogram contains a Lower Marker Peak, a System Peak and an Upper Marker Peak. The upper marker is 95% pure and contains two small impurities at 18 and 25 kD. The impurity level of the upper marker is corrected by the software for the concentration determination. Since these peaks are of the relatively low molecular weights, they do not interfere with the antibody (or half-antibody) peaks.

### EXAMPLE 1

### Methods for Determining, Sizing, Accuracy, and Reproducibility of Chip-Based Gel Electrophoresis

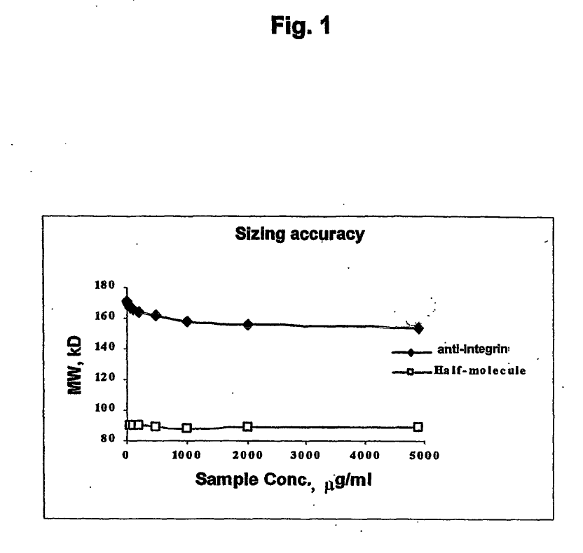

Analysis of the anti-integrin antibody samples identified one major peak corresponding to completely formed anti-integrin antibody (MW ˜160 kD), and a lower peak around ˜89 kD corresponding to the half-antibody. The same peak pattern was found for different anti-integrin antibody samples also treated under non-reducing conditions. The molecular weights of these two peaks for different samples are shown in Table 2, and the same data for anti-integrin antibody at different protein concentrations are shown in FIG. 1. While the data are highly reproducible, the molecular weight of the major peak decreases as the sample concentrations increase from 50 to 4900 μg/ml with an average MW of 161.1 for the intact anti-integrin antibody and 89.1 for the half-antibody

TABLE 2
Sizing Accuracy of Chip-Based Method.

| Antibody Samples | Antibody MW (kD) | Half-antibody MW(kD) |
|---|---|---|
| 1 | 156.3 | 88.9 |
| 2 | 154.7 | 89.3 |
| 12 | 161.1 | 90.8 |
| 5 | 157.4 | 90.1 |
| 6 | 156.5 | 90.8 |
| 3 | 157.9 | 90.8 |
| 4 | 157.9 | 90.8 |

### EXAMPLE 2

### Comparison of Chip-Based Gel Electrophoresis and SDS-PAGE

The half-antibody in anti-integrin samples are detectable by a non-reducing SDS-PAGE as a band that migrates to 80 kD. Data obtained by conventional SDS-PAGE was compared with the data obtained by the chip-based method. SDS-PAGE under non-reducing conditions was performed according to standard techniques (e.g., see Maniatis et al. (1995); Ausubel et al. (2001)). Typically for SDS-PAGE analysis under non-reducing conditions, usually 3 μg protein samples are loaded on the gel. For the chip-based method, as little as 1 μg is sufficient.

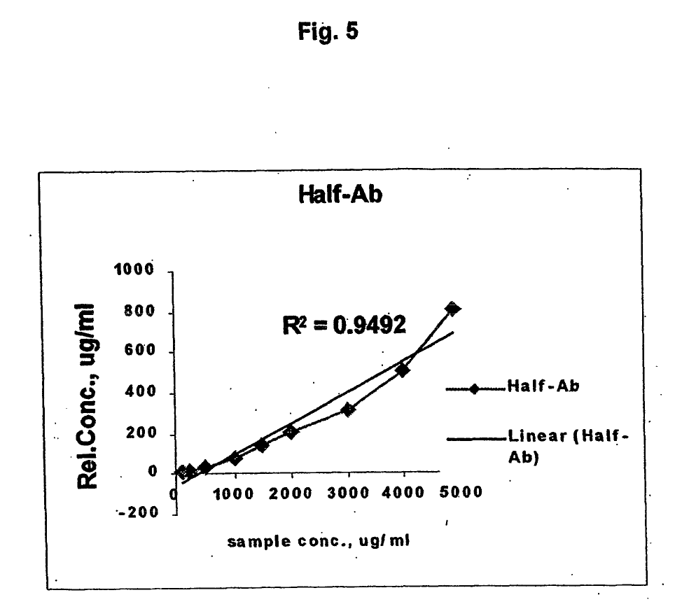

Subsequently, for some antibody samples, SDS-PAGE analysis was performed at the same low protein concentrations (or loaded amount of protein) as for the chip-based method in order to perform an accurate comparison (Table 3 and FIG. 5). These chip-based results demonstrate a good correlation between the two methods. Data obtained for a wide variety of antibody samples exhibiting a high percentage of half-antibodies, are presented in Table 3. For example, both SDS-PAGE and the chip-based method detected the high percentage of half-antibodies in the anti-integrin antibody samples 5, 8, 9 and 10 (Table 4). These results also demonstrate a good correlation between the two methods.

TABLE 3
Comparison of the Data Obtained by
Chip-Based Method and SDS-PAGE.

| Sample | | % Half-antibody | |
|---|---|---|---|
| protein Id, ug | conc., ug/ml | chip-based | SDS-PAGE |
| 1 | 3.8 | 11.7 | 10.8 |
| 0.8 | 3 | 10.7 | 10.2 |
| 0.6 | 2.3 | 11 | 9.3 |
| 0.5 | 1.9 | 10.8 | 8.6 |
| 0.4 | 1.5 | 10.1 | 13.5 |
| 0.3 | 1.1 | 9.3 | 8.6 |
| 0.2 | 0.8 | 9.8 | 10 |
| 0.1 | 0.4 | 11 | 6 |

Anti-integrin antibody sample 1 was analyzed at the different concentrations by the chip-based method (the percentage of half-antibodies was determined from the combined results table) and conventional SDS-PAGE.

TABLE 4
Comparison of the Data Obtained by the Chip-Based Method
and SDS-PAGE for Different Antibody Samples.

| | Protein | | Half- | |
|---|---|---|---|---|
| | loaded, μg | | Molecule, % | |
| Sample | chip-based | SDS-PAGE | chip-based | SDS-PAGE |
| 1 | 1 | 1 | 13.3 | 11.2 |
| | 0.5 | 0.5 | 10.9 | 9.3 |
| | 0.25 | 0.25 | 9.4 | 8.3 |
| 2 | 1 | 1 | 11.4 | 10.2 |
| | 0.5 | 0.5 | 10.9 | 9.2 |
| | 0.25 | 0.25 | 9 | 8.3 |
| 3 | 0.5 | 0.5 | 9.9 | 10.2 |
| | 0.25 | 0.25 | 8.3 | 10.7 |
| 4 | 0.5 | 0.5 | 10.3 | 12.7 |
| | 0.25 | 0.25 | 8.8 | 8.5 |
| 5 | 1 | 3 | 17.1 | 17.4 |
| | 0.5 | | 17 | |
| | 0.25 | | 15.3 | |
| | 0.12 | | 14.2 | |
| 6 | 1 | 3 | 13.7 | 12.5 |
| | 0.5 | | 12.9 | |
| | 0.25 | | 12.6 | |
| | 0.12 | | 9 | |
| 7 | 0.5 | 3 | 9.7 | 11.9 |
| | 0.27 | | 8.4 | |
| 8 | 0.5 | 3 | 20.5 | 23.8 |
| | 0.27 | | 19.1 | |
| 9 | 0.5 | 3 | 19 | 21.5 |
| | 0.27 | | 18 | |
| 10 | 0.5 | 3 | 14.6 | 17.4 |
| | 0.27 | | 14.2 | |
| 11 | 0.5 | 3 | 11.7 | 11.7 |
| | 0.27 | | 11.3 | |

Different antibody samples at the concentrations indicated were analyzed by the chip-based method and SDS-PAGE methods.

### EXAMPLE 3

### Methods to Determine the Linear Dynamic Range of the Assay

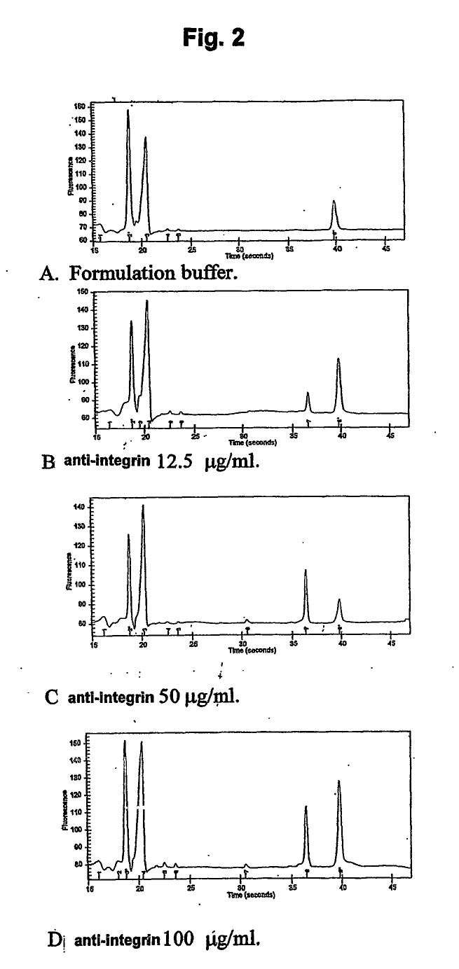

This example provides a method which can be used to determine the range of protein concentrations at which the results from this assay are accurate. To determine the linear dynamic range of the assay, anti-integrin antibody samples at concentrations from 12.5 to 4900 μg/ml were analyzed. FIG. 2 (panels A-D) show the results of analysis of different anti-integrin antibody samples at the lowest concentrations (12.5, 50 and 100 μg/ml) (B,C,D), and the control sample with the formulation buffer (A). The peak corresponding to the half-antibody band could be detected only at the total sample concentration of 50 μg/ml (C), although the intact antibody peak (MW˜171) can easily be seen at 12.5 μg/ml (B). With the staining approach used for the chip-based method, it is to be expected that, at some relatively high concentrations of protein, saturation in staining might be reached. An insufficient amount of dye might be present to bind in a quantitative fashion to the SDS-protein complexes. As a result the protein amount in the samples will be underestimated/overestimated. Accordingly it is important to identify the sample concentrations that give the linear correlation between the signal (the fluorescent intensity/relative concentration) and the “real” sample concentration. “Real” sample concentrations were determined by standard procedure based on A₂₈₀. These data are presented as the total sample concentration, and the concentrations corresponding to antibody and half-antibody (˜10%). Combined result table represents the different parameters of the assay results including fluorescence intensity (as ‘Corr.area”, relative concentration of the sample, and absolute concentration of the sample).

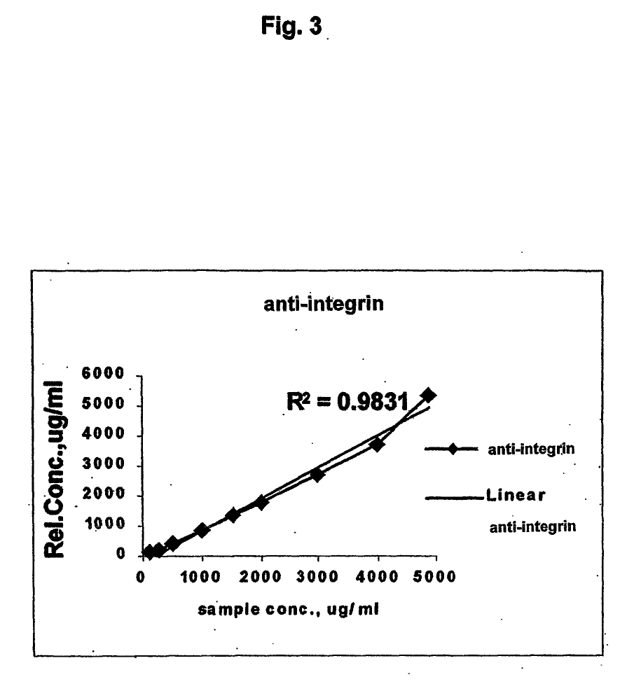

Experiments demonstrate that there was a lower correlation between the real sample concentration and fluorescence intensity, so the “relative sample concentration” was tested. Relative protein concentrations (μg/ml) are obtained based on measuring peak areas and comparing these measurements against the upper marker (myosin), which is used as an internal standard in each sample. The Agilent 2100 Bioanalyzer software automatically determines the peak area of the unknown proteins and the upper marker in each sample. The relative concentration of the unknown proteins within one sample is then calculated by the software based on the known concentration of the upper marker. The inclusion of the upper marker in each sample corrects for differences in sample injection into the separation channel and allows for reproducible quantitation. Real sample concentration was compared (“X”) to those determined by the software (relative and/or absolute) separately for the anti-integrin antibody and half-antibody. A sample of the reference standard (Sample 7) was diluted and tested as shown in FIGS. 3-6. The sample concentrations reported do not include the sample dilution by a factor of 15 in water before the sample is loaded in the chip.

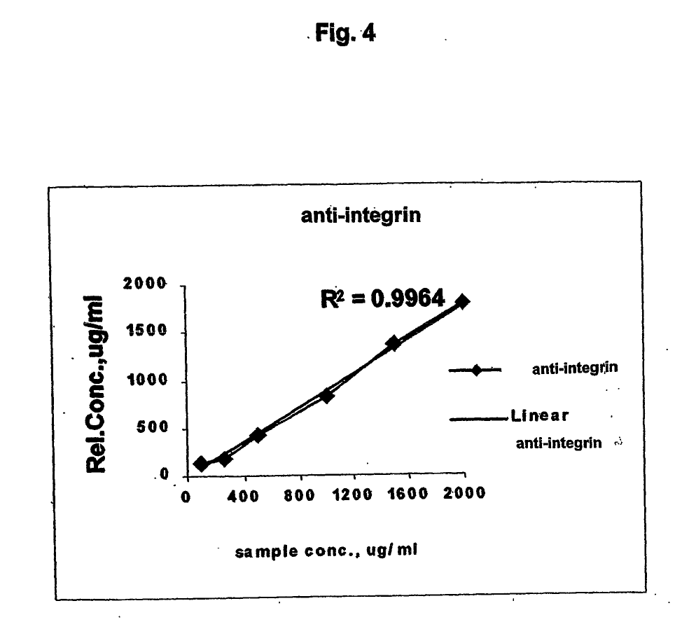

The assay is linear over two orders of magnitude, for example, from the lower detection limit, which is 12.5 μg/ml, up to 2000 μg/ml, based on the peak corresponding to the intact anti-integrin antibody (FIG. 4). The linearity is slightly but gradually declines past 2000 μg/ml (FIG. 5). A correlation coefficient of R²=0.98 was determined based on the intact antibody peak for the sample concentrations from 500 to 4900 μg/ml. At the concentrations from 100 to 2000 μg/ml, R²=0.996 was observed (FIG. 4).

### EXAMPLE 4

### Methods for Determining the Linear Dynamic Range of the Chip-Based Method

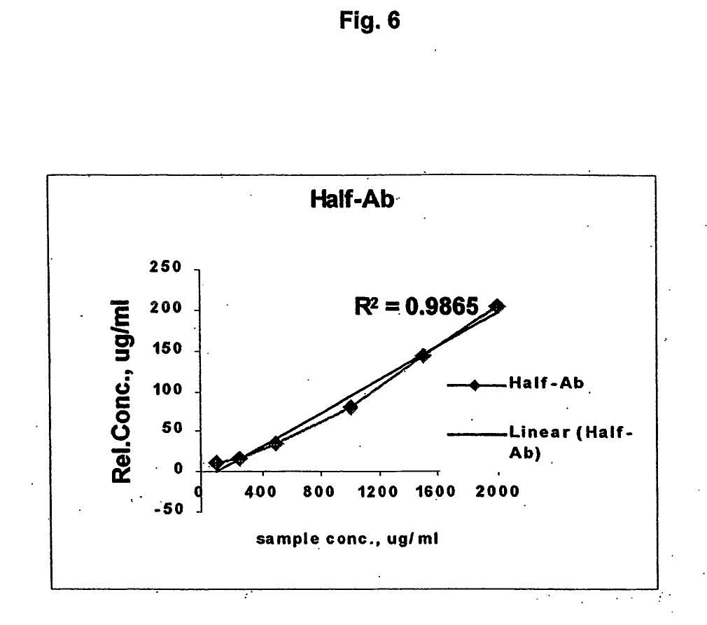

The following example provides a method by which the dynamic range of the chip-based method can be determined. The anti-integrin antibody standard (Sample 7) was analyzed at the protein concentrations 100, 200, 500, 1000, 1500, 2000, 3000, 4000, and 4900 μg/ml. Dilutions were made from the stock solution of the concentration 4900 μg/ml (concentration was determined based on A₂₈₀). The relative concentrations of the peaks corresponding to the intact antibody peak (FIGS. 3 and 4) and the half-antibody peak (FIGS. 5 and 6) were determined for a range of protein concentrations based on the combined results table (provided by software; Table 4) and a robust linear dynamic range was observed.

### EXAMPLE 5

### Methods for Determining the Correlation Between the Determined Absolute and Relative Antibody Concentrations and Total Antibody Sample Concentrations

The following example provides a method by which the actual and relative concentrations of a protein sample can be correlated. Two sets of antibody standards (sample 7) were analyzed at the following protein concentrations 1) 12.5, 25, 50, 100, 250, 500, 1000, 2000, and 4900 μg/ml and 2) 100, 200, 500, 1000, 1500, 2000, 3000, 4000, and 4900 μg/ml. Dilutions were made from a stock solution (4900 μg/ml; concentration was determined based on A₂₈₀). Samples at all concentrations except 4900 μg/ml, were used for the calibration curve for the absolute concentration determination. Total relative and absolute concentrations were determined from the combined results table.

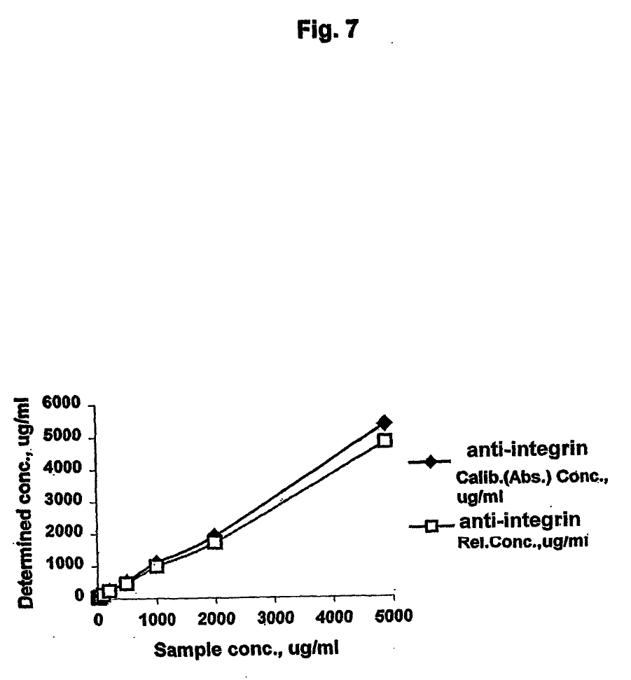

The same graph based on the half-antibody peak demonstrates the linear dynamic range for samples at the concentrations from 50 to 2000 μg/ml. A correlation coefficient of R²=0.949 was determined for the sample concentrations from 100 to 4900 μg/ml (FIG. 5). At the concentrations from 100 μg/ml to 2000 μg/ml a correlation coefficient of R²=0.987 (FIG. 6) was observed. Lower concentrations were not tested because the preliminary data already showed the absence of the half-antibody peak at 10-20 μg/ml. FIG. 7 demonstrates a good correlation between the relative and absolute concentrations of samples determined by software analysis, and the real sample concentration determined based on A₂₈₀ measurement. At low total sample concentrations, the % half-antibody (the relative concentration of the half-antibody) is less than the % half-antibody obtained at the higher sample concentration (within a linear range).

### EXAMPLE 6

### Methods for Determining the Optimum Sample Concentration

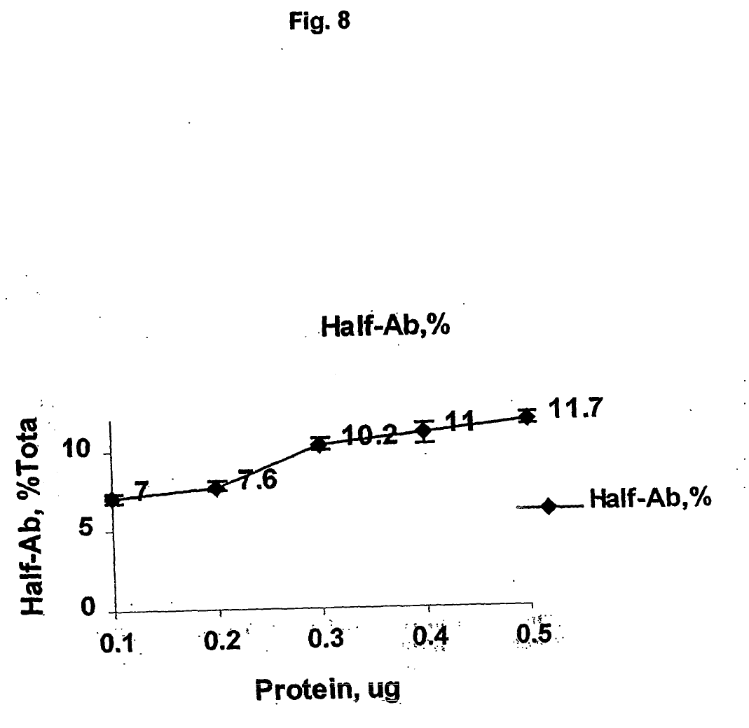

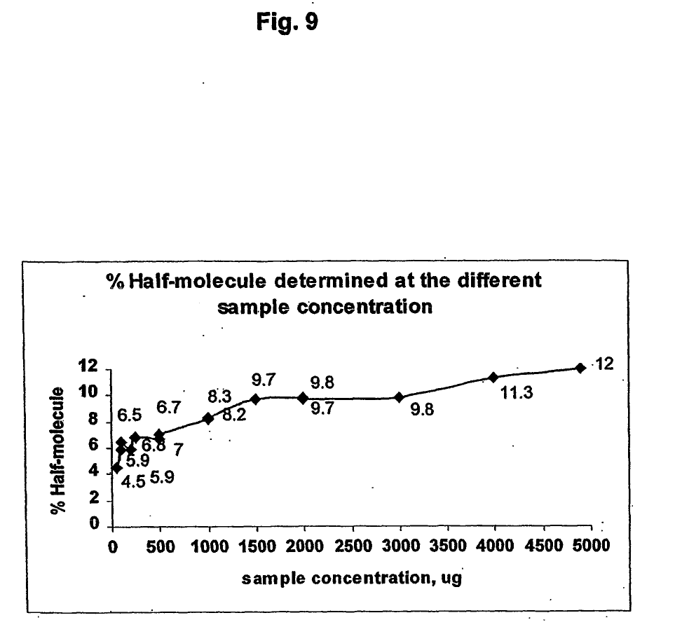

The following example provides a method to determine the optimum protein concentration that should be used to obtain optimal results. To determine the optimum sample concentration, the results of % half-antibody obtained at different sample concentrations were analyzed (FIGS. 8 and 9). The % half-antibody determined at lower concentrations are significantly lower (≦1000 μg/ml). Based on these results the recommended optimum sample concentration is 3000 μg/ml, however, reproducible results were achieved using samples from 500 μg/ml to 5000 μg/ml.

### EXAMPLE 7

### Methods for Determining of the Limit of Detection (LOD)

The following example provides a method for determining the lowest concentration at which half-antibodies are accurately detected. The limit of detection was determined based on the observations that the lowest detected concentration for half-antibody is 2.7 μg/ml at the sample concentration 50 μg/ml. This determined concentration resulted in the low % half-antibody detected (4.5 % compared with the average 10%, see below) thus, the real concentration of the half-antibody in the sample can be twice as much (˜5 μg/ml). Accordingly, the optimum sample concentration suggested for this assay is ˜3000 μg/ml. Based on this assumption, the limit of quantitation for the % half-antibody can be determined as the half-antibody concentration of5 μg/ml (2 ng loaded), and 0.17%—(5 μg/ml/3000 μg/ml). As the % half-antibody is ˜10%, it corresponds to a sample concentration of 50 μg/ml.

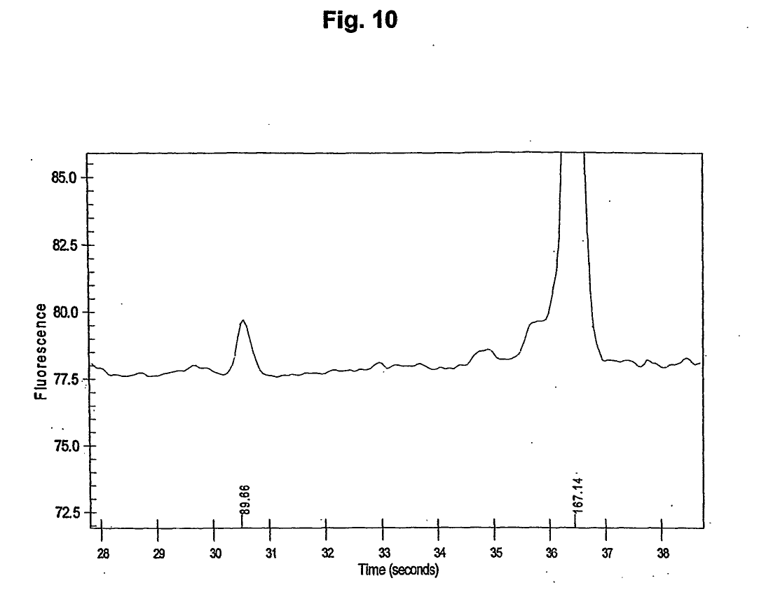

The calculations were done based on relative concentration data. FIG. 10 shows raw data at 50 μg/ml. The half-antibody peak with a MW 89.66 is clearly distinguished from the background.

### EXAMPLE 8

### Methods for Determining the Limit of Quantitation (LOQ)

The following example provides a method by which the maximum concentration at which half-antibody determination is reliable. Specifically, to determine the limit of quantitation, the % half-antibody determined at different sample concentrations were compared. The LOQ is determined to be a 613 μg/ml sample concentration, and/or 1.9%; for samples prepared with a sulfhydryl alkylating agent, an LOQ of 0.59% was achieved. This was calculated based on the assumption that, the optimum sample concentration is 3000 μg/ml and the average % half-antibody for the antibody reference standard is 9. 1%. Linear regression was applied for the points (sample concentrations) that gives the % half-antibody 20% higher or lower than the average (7.3-11%).

### EXAMPLE 9

### Methods for Determining the Repeatability and Precision of the Chip-Based Method

The following example provides a method to test the reproducibility and precision of the chip-based assay. The reproducibility of the assay was tested by using the same sample and the same chip, when using multiple chips, or when using the same sample with different chips. The antibody reference standard (sample 7) was used to verify the repeatability of the chip-based method. Data obtained from running the same sample on the same chip are shown in Table 5. The data (the % half-antibody, which is “% Total” determined from the combined results table) are very similar with STDEV=0.5 and % STDEV=0.5 and 6.0, respectively, for antibody and half-antibody.

TABLE 5
Comparison Data for Different Samples Run on the Same Chip

| Antibody, % | | Half-antibody, % |
|---|---|---|
| | 90.6 | 9.4 |
| | 92.0 | 8.0 |
| | 92.3 | 7.7 |
| | 91.8 | 8.2 |
| | 91.8 | 8.2 |
| | 91.5 | 8.5 |
| | 91.4 | 8.6 |
| | 91.8 | 8.2 |
| | 91.2 | 8.8 |
| | 91.8 | 8.2 |
| Average | 91.6 | 8.4 |
| STDEV | 0.5 | 0.5 |
| % STDEV | 0.5 | 6.0 |

Data analysis of the same sample run on different chips are shown in Table 6. The average for the % half-antibody determined at 0.1, 0.2, 0.3, 0.4 and 0.5 μg is 9.5, with STDEV 0.3, and % STDEV 3.36.

TABLE 6
Comparison Data for the Same Sample Run on Different Chips

| | Load, ug | | | | |
|---|---|---|---|---|---|
| | 0.5 | 0.4 | 0.3 | 0.2 | 0.1 |
| Half- | 11.8 | 10.8 | 10 | 6.9 | 6.7 |
| molecule, % | | | | | |
| | 11.5 | 11.5 | 10.7 | 7.2 | 7.1 |
| | 11.5 | 10.9 | 10.1 | 8.2 | 7.4 |
| | 11.4 | 11 | 10 | 7.6 | 7 |
| | 12 | 11 | 10.1 | 7.8 | 7 |
| | 12 | 11 | 10.2 | 7.6 | 6.8 |
| Average | 11.7 | 11 | 10.2 | 7.6 | 7 |
| STDEV | 0.3 | 0.2 | 0.3 | 0.5 | 0.2 |
| % STDEV | 2.6 | 1.8 | 2.9 | 6.6 | 2.9 |

An antibody sample (i.e., number 12) was loaded in amounts of 0.1, 0.2, 0.3, 0.4 and 0.5 μg (concentrations 370, 750, 1100, 1500 and 1870 μg/ml) to determine the % half-antibody, and antibody. It was also observed that the assay can tolerate a large variety of sample buffers and additives.

### EXAMPLE 10

### Methods for Determining the Repeatability and Precision of the Chip-Based Method for Detecting Impurities

The following example provides a method to test the reproducibility and precision of the chip-based assay for detecting impurities in a sample, for example, in a sample containing a recombinant antibody.

The reproducibility of the assay was tested by using several recombinant antibody samples having, for example, either known marker proteins of a certain concentration that were intentionally added, or, residual impurities from a standard recombinant protein preparation and recovery process that were detected by other means (e.g., SDS-PAGE).

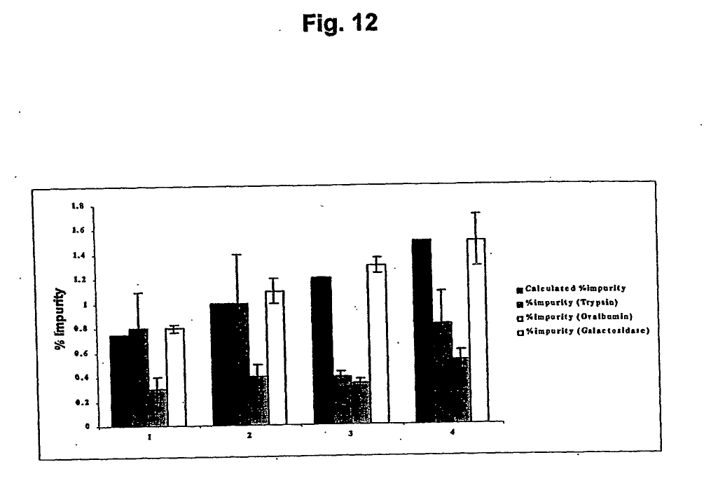

In one experiment, three marker proteins (trypsin inhibitor (MW 21 kD), ovalbumin (MW 46 kD), and galactosidase (MW 116 kD) were added to a recombinant antibody reference standard (i.e., natalizumab) so that the final concentration of the sample was 4800 μg/ml, and the final concentrations of the marker proteins were correspondent to 0.75%, 1%, 1.2%, and 1.5% impurity. The data of the recovery are shown in Table 7 and FIG. 12.

TABLE 7
Recovery of Impurities in a Recombinant Protein Sample

| | Trypsin | | |
|---|---|---|---|
| | Inhibitor | Ovalbumin | Galactosidase |
| Calculated % Impurity | 0.75 | 0.75 | 0.75 |
| Determined % Impurity | 0.8 | 0.3 | 0.8 |
| Average (n = 11) | | | |
| STDEV | 0.3 | 0.1 | 0.03 |
| % STDEV | 38 | 33 | 4 |
| Calculated % Impurity | 1 | 1 | 1 |
| Determined % Impurity | 1 | 0.4 | 1.1 |
| Average (n = 11) | | | |
| STDEV | 0.4 | 0.1 | 0.1 |
| % STDEV | 40 | 25 | 9 |
| Calculated % Impurity | 1.2 | 1.2 | 1.2 |
| Determined % Impurity | 0.39 | 0.34 | 1.3 |
| Average (n = 11) | | | |
| STDEV | 0.05 | 0.03 | 0.06 |
| % STDEV | 13 | 9 | 5 |
| Calculated % Impurity | 1.5 | 1.5 | 1.5 |
| Determined % Impurity | 0.82 | 0.53 | 1.5 |
| Average (n = 11) | | | |
| STDEV | 0.27 | 0.08 | 0.21 |
| % STDEV | 33 | 15 | 14 |

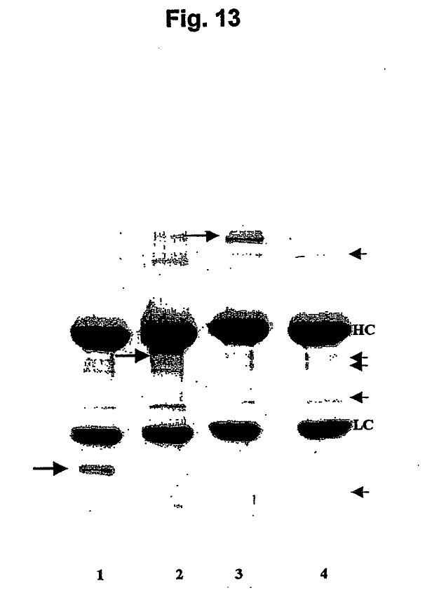

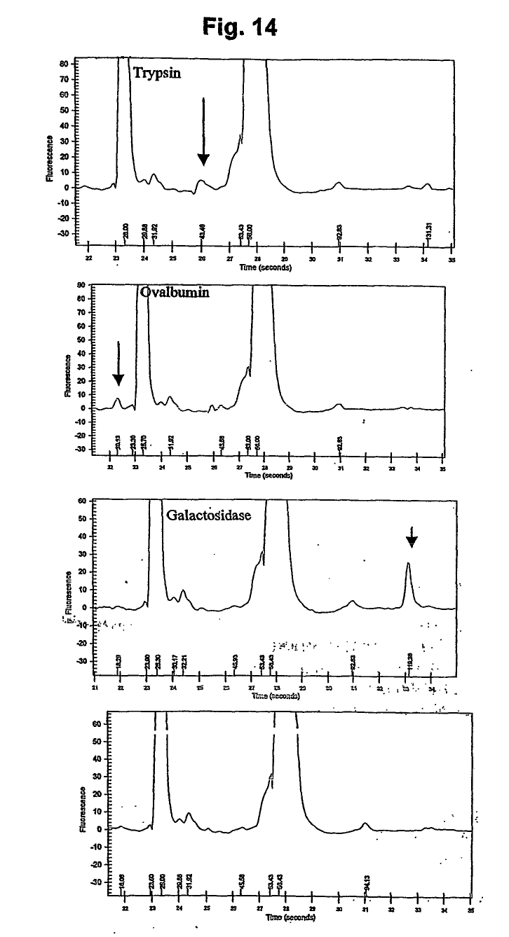

To determine the sensitivity of the chip-based method, a comparison of impurity analysis data obtained by SDS-PAGE and the chip-based method was performed (FIGS. 13 and 14). A reducing SDS-PAGE indicated that the chip-based method had a limit of detection (LOD) of 0.1%, and limit of quantitation (LOQ) of 0.5%.

Accordingly, it was concluded that the chip-based method is an efficient and reliable approach for detecting and quantitating impurities in a polypeptide sample, e.g., a sample containing a recombinant protein such as an antibody.

### EXAMPLE 11

### Methods for Determining the Inhibitory Activity of Sulfhydryl Alkylating Agents on Polypeptide Degradation

The following example provides a method to test the inhibitory activity of sulfhydryl alkylating agents on polypeptides, for example, polypeptide multimers, such as antibodies.

It had been observed that the denaturing of proteins by heating prior to performing non-reducing SDS-PAGE can result in degradation and liberation of polypeptide fragments. Based on the profile of the polypeptide fragments it was concluded that heat treatment resulted in peptide bond hydrolysis as well as reduction of disulfide bonds. It was also determined using DTNB (5,5′-dithio-bis(2-nitrobenzoic acid) titration, that only 1 mole of thiol groups appeared upon heat treatment, not 2 as expected if disulfide bonds were reduced. This process is inhibited by iodoacetamide that can interact with free thiol groups. Conclusions from this study indicate that the heat-induced liberation of polypeptide fragments is a result of a thiol-disulfide interchange reaction triggered by the thiol residue exposed by rupture of the thioester bond.

To determine the inhibitory activity of sulfhydryl alkylating agents, e.g., iodoacetamide and, in particular, N-ethylmaleimide (NEM) on polypeptide degradation, a modified chip-based gel electrophoresis method was used. As a test polypeptide, a recombinant polypeptide multimer, in particular, an anti-integrin antibody was used at several different sample concentrations, e.g., at 2 mg/ml and 5 mg/ml. The assay for detecting the polypeptide, and by-products thereof, e.g., half-antibody products as well as other impurities, was determined to have a high degree of reproducibility (e.g., a LOD score of 0.1% and 0.05% for 2 mg/ml load and 5 mg/ml load, respectively, was observed and a LOQ score for half-antibody of ≦0.5%).

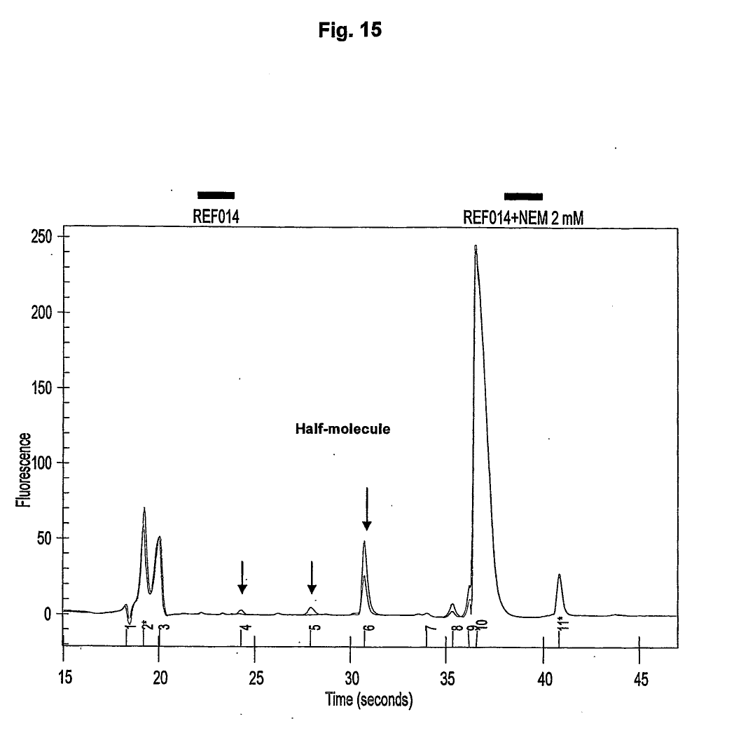

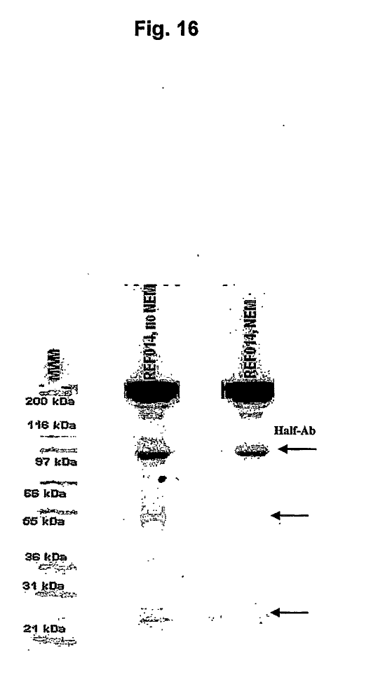

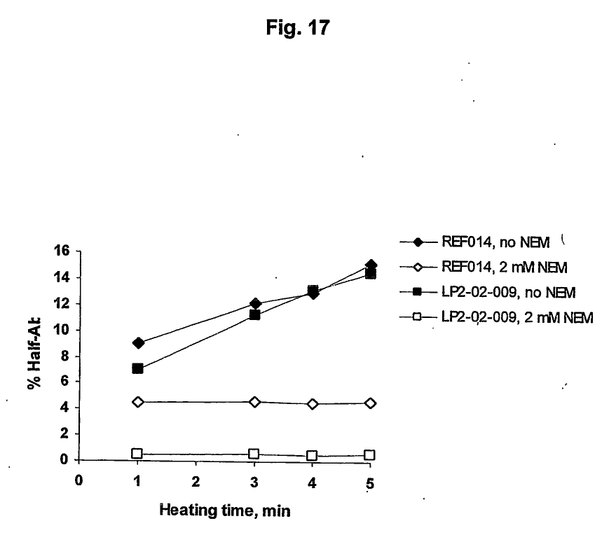

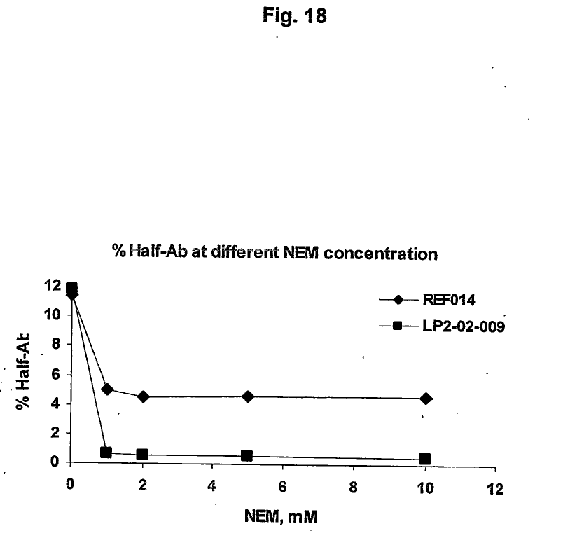

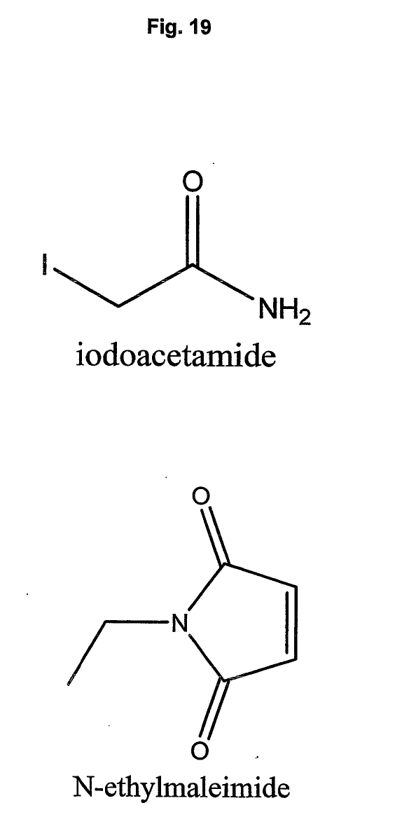

The chip-based gel electrophoresis method used in this example is similar to the method as described in the foregoing examples except that the sample preparation step has been modified. Indeed, the polypeptide sample is prepared with a sulfhydryl alkylating agent such as iodoacetamide or N-ethylmaleimide (see FIG. 19). Polypeptide samples prepared in this fashion had remarkably fewer by-products. In particular, the percentage of half-antibody decreased. For example, when a polypeptide multimer, such as an anti-integrin antibody was tested the percentage of half-antibody is 5% in the presence of NEM, and 9% without NEM (see FIG. 15). This difference was also confirmed using standard SDS page (see FIG. 16). In addition, the difference in inhibitory activity between polypeptide samples treated and untreated when exposed to heat over time was also examined. Results demonstrate that the longer a polypeptide is exposed to heat, the greater the prevalence of by-products, for example, half-antibodies as well as other impurities. This accumulation of by-products, however, is blocked when the polypeptide sample comprises an inhibitor of polypeptide degradation (see FIG. 17). In a related experiment, this inhibitory activity of polypeptide degradation was tested over a range of concentrations from 1 mM to 10 mM (see FIG. 18). Typically, a concentration as low as 2 mM showed reliable inhibitory activity.

Accordingly, it was concluded that the chip-based method is an efficient and reliable approach for detecting and quantitating impurities in a polypeptide sample, e.g., a sample containing a recombinant protein such as an antibody, and that the presence of half-antibodies as well polypeptide fragments and impurities, can be greatly reduced using an inhibitor of disulfide bond rearrangement as a result of polypeptide degradation, e.g., a sulfhydryl alkylating agent, e.g., N-ethylmaleimide (NEM).

### Equivalents

For one skilled in the art, using no more than routine experimentation, there are many equivalents to the specific embodiments of the invention described herein. Such equivalents are intended to be encompassed by the following claims.

1. A method for detecting the presence of a IgG4 polypeptide having a selected disulfide linkage pattern in a sample comprising, loading a sample containing a polypeptide having a selected disulfide linkage pattern, wherein the sample comprises an inhibitor of disulfide bond rearrangement, onto a chip comprising a channel having a separation medium effective to act as an obstacle to the migration of the polypeptide having a selected disulfide linkage pattern, and at least two electrodes disposed within the channel to induce an electric field, applying an electric field across the separation medium of the chip whereby a separation of the IgG4 polypeptide having a selected disulfide linkage pattern as compared to a IgG4 polypeptide not having the selected disulfide linkage pattern is achieved, and determining the presence of the IgG4 polypeptide having a selected disulfide linkage pattern.

2. A method for detecting the presence of a polypeptide having a selected disulfide linkage pattern in a sample consisting of a mixture of polypeptide multimers having two or more polypeptide chains and comprising at least one disulfide linkage between the polypeptide chains comprising, loading a sample containing the mixture of polypeptide multimers, wherein the sample comprises an inhibitor of disulfide bond rearrangement, onto a chip comprising a channel having a separation medium effective to act as an obstacle to the migration of the polypeptide having a selected disulfide linkage pattern, and at least two electrodes disposed within the channel to induce an electric field, applying an electric field across the separation medium of the chip whereby a separation of the polypeptide having a selected disulfide linkage pattern as compared to a polypeptide not having the selected disulfide linkage pattern is achieved, and determining the presence of the polypeptide having a selected disulfide linkage pattern.

3. The method of claim 1, wherein the inhibitor is a sulfhydryl alkylating reagent.

4. The method of claim 3, wherein the sulfhydryl alkylating reagent is selected from the group consisting of iodoacetamide and N-ethylmaleimide (NEM).

5. The method of claim 4, wherein the sulfhydryl alkylating reagent is N-ethylmaleimide (NEM).

6. The method of claim 5, wherein the amount of N-ethylmaleimide (NEM) is between about to about 10 mM.

7. The method of claim 1, wherein the method further comprises determining the presence of a polypeptide impurity.

8. The method of claim 1, wherein the IgG4 polypeptide having a selected disulfide linkage pattern is a half-antibody.

9. The method of claim 2, wherein the polypeptide having a selected disulfide linkage is a half-antibody.

10. The method of claim 9, wherein the half-antibody is of the IgG4 class.

11. The method of claim 1, wherein the IgG4 polypeptide having a selected disulfide linkage pattern is recombinantly produced.

12. The method of claim 1, wherein the polypeptide is recombinantly produced.

13. The method of claim 1, wherein the polypeptide having a selected disulfide linkage pattern is recombinantly produced.

14. The method of claim 1, wherein, the IgG4 polypeptide not having the selected disulfide linkage pattern is an anti-integrin antibody.

15. The method of claim 2, wherein the mixture comprises an anti-integrin antibody.

16. The method of claim 14, wherein the anti-integrin antibody is recombinantly produced.

17. The method of claim 1, wherein the sample is obtained from the growth medium of a cell culture.

18. The method of claim 1, wherein the sample comprises about 1 to about 5000 ug/ml of a polypeptide having a selected disulfide linkage pattern.

19. The method of claim 1, wherein the separation medium is a gel polymer.

20. The method of claim 1, wherein the separation medium is non-reducing.

21. The method of claim 1, wherein the migration of the polypeptide is detected using a fluorescence detector.

22. The method of claim 1, wherein the electric field is non-alternating.

23. The method of claim 1, wherein the separation further comprises isoelectric focusing.

24. The method of claim 1, wherein the separation is according to the molecular weight of the polypeptide.

25. The method of claim 1, wherein the chip comprises a precast gel polymer.

26. A kit for detecting the presence of a polypeptide having a selected disulfide linkage pattern comprising, a chip and instructions for carrying out the method of claim 1.

27. A kit for determining the purity of a therapeutic polypeptide having a selected disulfide linkage pattern comprising, a chip and instructions for carrying out the method of claim 1.

28. The kit of claim 26, wherein the kit further comprises a component selected from the group consisting of, separation medium, non-reducing buffer, protein dye, formulation buffer, and means for inducing an electric field through a separation medium.

29. The kit of claim 26, wherein the kit further comprises instructions for determining the presence of a polypeptide impurity.

30. The kit of claim 26, wherein the kit further comprises one or more polypeptide standards.

31. A method of inhibiting disulfide bond rearrangement, wherein the polypeptide is incubated with a sulfhydryl alkylating agent selected from the group consisting of iodoacetamide and N-ethylmaleimide (NEM).

32. The method of claim 31 wherein the sulfhydryl alkylating reagent is N-ethylmaleimide (NEM).

33. The method of claim 32, wherein the concentration of N-ethylmaleimide (NEM) is between about 1 mM to about 10 mM.

34. The method of claim 31 wherein the disulfide bond rearrangement occurs upon exposure to heat.

35. A composition comprising a polypeptide and inhibitor of disulfide bond rearrangement, wherein the inhibitor is a sulfhydryl alkylating agent.

36. The composition of claim 35, wherein the sulfhydryl alkylating agent is selected from the group consisting of iodoacetamide and N-ethylmaleimide (NEM).

37. The composition of claim 36, wherein the sulfhydryl alkylating reagent is N-ethylmaleimide (NEM).

38. The composition of claim 37, wherein the concentration of N-ethylmaleimide is between about 1 to about 10 mM.

39. The composition of claim 35, wherein the polypeptide is a multimeric polypeptide.

40. The composition of claim 39, wherein multimeric polypeptide is an antibody or half-antibody.

41. The composition of claim 40, wherein the antibody is an IgG4 antibody.

42. The composition of claim 41, wherein the antibody is an anti-integrin antibody.
